(19) United States
(12) Patent Application Publication
(10) Pub. No.: US 2009/0028785 A1
(43) Pub. Date: Jan. 29, 2009

(54) MEDICAL DEVICES WITH COATINGS FOR DELIVERY OF A THERAPEUTIC AGENT

(75) Inventor: John T. Clarke, Kiniska (IE)

Correspondence Address: John J. Gagel;C/O Fish & Richardson, 225 Franklin Street, Boston, MA, 02110-2804, US

(73) Assignee: Boston Scientific Scimed, Inc., Maple Grove, MN (US)

(21) Appl. No.: 12/175,487

(22) Filed: Jul. 18, 2008

Related U.S. Application Data
(60) Provisional application No. 60/951,280, filed on Jul. 23, 2007.

(51) Int. Cl. A61F 2/82; A61K 51/00; A61K 31/436; A61K 31/337

(52) U.S. Cl. 424/1.11; 424/423; 514/449; 514/294

(57) ABSTRACT

Described herein are implantable coated medical devices, such as intravascular stents, for delivering therapeutic agents to the body tissue of a patient, and methods for making such medical devices. In particular, described herein are implantable coated medical devices comprising a substrate having a surface, and a coating disposed upon the surface that comprises a coating composition that includes a releasable metal oxide. The coating is free of polymer or a particular type of polymer that is not a part of any releasable metal oxide.

This application claims priority to U.S. Provisional Application No. 60/951,280 filed on Jul. 23, 2007, which is incorporated herein by reference in its entirety.

### 1.0 INTRODUCTION

Described herein are implantable coated medical devices, such as intravascular stents, for delivering therapeutic agents to the body tissue of a patient, and methods for making such medical devices. In particular, described herein are implantable coated medical devices comprising a substrate having a surface, and a coating disposed upon the surface that comprises a coating composition that includes a releasable metal oxide. The coating is free of polymer or a particular type of polymer that is not a part of any releasable metal oxide.

### 2.0 BACKGROUND

Medical devices have been used to deliver therapeutic agents locally to the body tissue of a patient. For example, stents having a coating containing a therapeutic agent, such as an anti-restenosis agent, have been used in treating or preventing restenosis. Currently, such medical device coatings include a therapeutic agent alone or a combination of a therapeutic agent and a polymer. Both of these types of coatings may have certain limitations.

Coatings containing a therapeutic agent without a polymer are generally ineffective in delivering the therapeutic agent since such coatings offer little or no control over the rate of release of the therapeutic agent. Specifically, the therapeutic agent is generally delivered in a burst release within a few hours. Therefore, many medical device coatings include a therapeutic agent and a polymer to provide sustained release of the therapeutic agent over time.

Though the use of polymers in coatings can provide control over the rate of release of the therapeutic agent therefrom, the use of such polymers in coatings may present certain other limitations. For example, the polymer in the coating may react adversely with the blood and cause thrombosis.

Moreover, some polymer coating compositions do not actually adhere to the surface of the medical device. In order to ensure that the coating compositions remain on the surface, the area of the medical device that is coated, such as a stent strut, is encapsulated with the coating composition. However, since the polymer does not adhere to the medical device, the coating composition is susceptible to deformation and damage during loading, deployment and implantation of the medical device. Any damage to the polymer coating may alter the therapeutic agent release profile and can lead to an undesirable increase or decrease in the therapeutic agent release rate.

Also, surfaces coated with compositions comprising a polymer may be subject to undesired adhesion to other surfaces. For instance, balloon expandable stents must be put in an unexpanded or “crimped” state before being delivered to a body lumen. During the crimping process coated stent struts are placed in contact with each other and can possibly adhere to each other. When the stent is expanded or uncrimped, the coating on the struts that have adhered to each other can be damaged, torn-off or otherwise removed. Moreover, if the polymer coating is applied to the inner surface of the stent, it may stick or adhere to the balloon used to expand the stent when the balloon contacts the inner surface of the stent during expansion. Such adherence to the balloon may prevent a successful deployment of the medical device.

Similar to balloon-expandable stents, polymer coatings on self-expanding stents can also interfere with the delivery of the stent. Self-expanding stents are usually delivered using a pull-back sheath system. When the system is activated to deliver the stent, the sheath is pulled back, exposing the stent and allowing the stent to expand itself. As the sheath is pulled back it slides over the outer surface of the stent. Polymer coatings located on the outer or abluminal surface of the stent can adhere to the sheath as it is being pulled back and disrupt the delivery of the stent.

Accordingly, there is a need for medical devices and coatings for medical devices that have little or no polymer and that can release an effective amount of a therapeutic agent in a controlled release manner while avoiding the disadvantages of current coatings for medical devices that include a polymer. Additionally, there is a need for methods of making such medical devices and coatings for medical devices.

### 3.0 SUMMARY

These and other objectives are addressed by the embodiments described herein. In certain embodiments, coatings for medical devices that are capable of releasing a therapeutic agent in a controlled release manner as well as methods for making such devices.

For instance, in one embodiment, an implantable coated medical device, such as a stent, comprises a substrate having a surface. A coating is disposed on at least a portion of the surface, in which the coating is free of any synthetic polymer, or in some instances free of any polymer, that is not part of any releasable metal oxide. The coating comprises a first coating composition disposed on at least a portion of the surface. The first coating composition comprises a releasable metal oxide, and in certain instances, a first therapeutic agent. In some embodiments, the coating further comprises a second coating composition disposed on the surface. The second coating composition comprises a releasable metal oxide and is disposed between the surface and the first coating composition. The second coating composition may, but need not include, a therapeutic agent.

In another embodiment, an implantable coated medical device, such as a stent, comprises a substrate having a surface. A coating is disposed on at least a portion of the surface, in which the coating is free of any synthetic polymer, or in some instances free of any polymer, that is not part of any releasable metal oxide. The coating comprises a first coating composition disposed on at least a portion of the surface. The first coating composition comprises a non-releasable metal oxide having a plurality of pores therein. In certain instances, a first therapeutic agent maybe disposed in at least some of the pores of the non-releasable metal oxide. A second coating composition comprising a releasable metal oxide, and in some instances a therapeutic agent, is disposed on at least a portion of the first coating composition. In some instances, the pores of the non-releasable metal oxide are free of any therapeutic agent before the coating composition is disposed on the first coating composition.

In yet another embodiment, an implantable coated medical device, such as a stent, comprises a substrate having a surface. The substrate comprises a non-releasable metal oxide having a plurality of pores therein. In some instances, a first therapeutic agent may be disposed in at least some of the pores of the non-releasable metal oxide. A coating is disposed on at least a portion of the surface, in which the coating is free of any synthetic polymer, or in some instances, free of any polymer, that is not part of any releasable metal oxide. The coating comprises a coating composition comprising a releasable metal oxide and in some instances a therapeutic agent. In certain embodiments, the pores of the non-releasable metal oxide are free of any therapeutic agent before the second coating composition is disposed on the surface.

### 3.1 DEFINITIONS

As used herein “synthetic polymer” refers to polymers that are man-made or not naturally occurring.

As used herein, a coating that is “free of any polymer or any synthetic polymer” means that no polymer or synthetic polymer was purposefully or intentionally added to the materials used to make the coating.

As used herein, “metal oxide” refers to a chemical compound in which oxygen is combined with one or more metals.

As used herein, “releasable metal oxide” refers to a metal oxide that can become released from the medical device, e.g. a coating of a medical device, when the medical device is implanted in a patient. The metal oxide can dissolve or dissociate into small oxide particles and/or release molecules that are bonded to the surface of the oxide. For example, a metal oxide coating and/or the molecules bonded to the metal oxide can be released by being exposed to body fluid or tissue that dissolves, dissociates or otherwise facilitates the release of the metal oxide and/or the molecules bonded to the metal oxide coating.

As used herein, “polymer that is not part of any releasable metal oxide” refers to a polymer that is not chemically bonded directly or indirectly to a releasable metal oxide.

As used herein, “non-releasable metal oxide” refers to a metal oxide that does not become released from the medical device, e.g. a coating of a medical device, when the medical device is implanted in a patient.

As used herein an “organic group” refers to an organic chemical moiety.

As used herein a “adduct of a metal oxide and organic group” refers to a chemical compound comprising at least one metal oxide that is bound to at least one organic group, by for example hydrogen bonding.

As used herein, “pores” refers to openings or voids.

As used herein, “free of a therapeutic agent” or “free of any therapeutic agent” means that no therapeutic agent was purposefully or intentionally included.

As used herein “about” is synonymous with the term “approximately,” and refers to a little more or less than the stated value.

As used herein, the terms “controlled release,” “sustained release”, “modulated release” and “modified release” can be used interchangeably and are used to describe the release profile of a therapeutic agent that is not an immediate or burst release profile.

### 4.0 BRIEF DESCRIPTION OF THE DRAWINGS

Certain embodiments will be explained with reference to the following drawings.

FIGS. 1A-1B show cross-sectional views of examples of medical devices having a substrate and a coating comprising a releasable metal oxide.

FIGS. 2A-2B show cross-sectional views of examples of medical devices having a substrate and a coating disposed on the surface of the substrate, in which the coating comprises a first coating composition comprising a releasable metal oxide and a second coating comprising a non-releasable metal oxide.

FIGS. 3A-3B show cross-sectional views of examples of medical devices having a substrate and a coating comprising a releasable metal oxide disposed on the surface of the substrate.

FIGS. 4A-4B show cross-sectional views of two other embodiments of coated medical devices.

FIG. 5 shows a peripheral view of an embodiment of an intravascular stent.

FIG. 6A-6B show the therapeutic agent release profiles for various coated samples prepared according to Example 1.

### 5.0 DETAILED DESCRIPTION

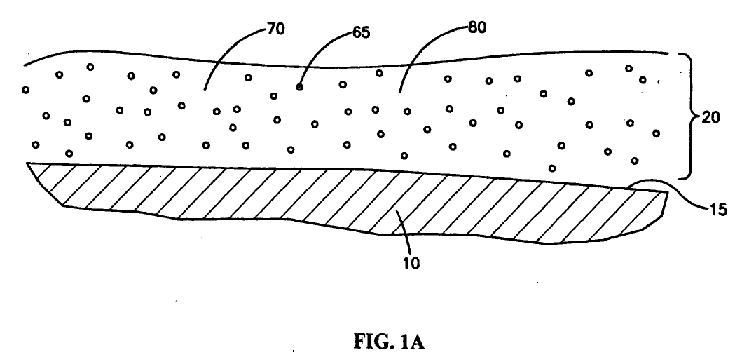

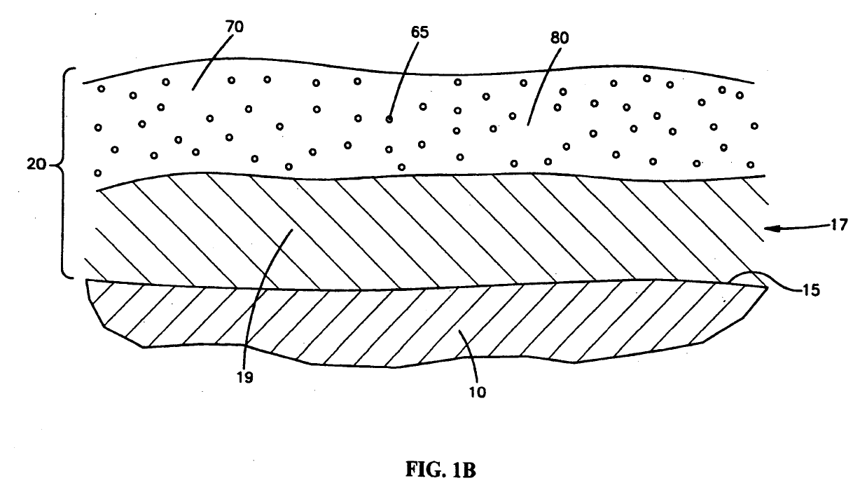

In one embodiment, the medical devices have a substrate having a surface; and a coating disposed on at least a portion of the surface. The coating is free of any synthetic polymer, or in certain instances is free of any polymer, that is not part of any releasable metal oxide. FIG. 1A shows a cross-sectional view of an example of such an embodiment. In this example, the substrate 10 of the medical device, which can be a stent, has a surface 15 that is coated with coating 20. The coating 20 comprises a first coating composition 70 that is disposed on at least a portion of the surface 15. The first coating composition 70 comprises a first releasable metal oxide 80 and a therapeutic agent 65. In some embodiments, the first releasable metal oxide 80 and the therapeutic agent 65 are distributed throughout the first coating composition 70. In other embodiments, the first releasable metal oxide 80 and the therapeutic agent 65 can be located in discrete parts of the coating composition. In some embodiments, before it is applied to the medical device, the releasable metal oxide can comprise the therapeutic agent. For example, the releasable metal oxide can be an adduct of a metal oxide and an organic group that can comprise a therapeutic agent, e.g., the therapeutic agent is part of the adduct by being hydrogen bonded to a part of the adduct. When the medical device is implanted in a patient, the first releasable metal oxide 80 is released from the medical device along with the therapeutic agent 65. In some figures, provided herein, therapeutic agents are represented as black dots; however, such representations should not be interpreted as requiring the therapeutic agents to exist only in particulate form.

FIG. 1B shows an embodiment that is similar to that shown in FIG. 1A. In addition to the first coating composition 70, the coating 20 includes a second coating composition 17 that comprises a second releasable metal oxide 19. The second coating composition 17 is disposed between the first coating composition 70 and the substrate surface 15. The first releasable metal oxide 80 and the second releasable metal oxide 19 can be the same or different. Also, the second coating composition 17 can also comprise a therapeutic agent, which can be the same as or different from the therapeutic agent 65 of the first coating composition 70. In some embodiments, before it is applied to the medical device, the releasable metal oxide can comprise the therapeutic agent.

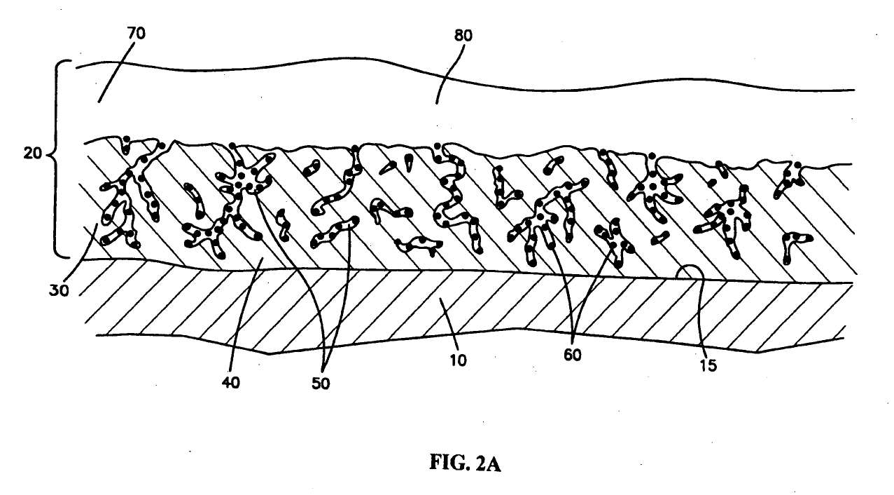

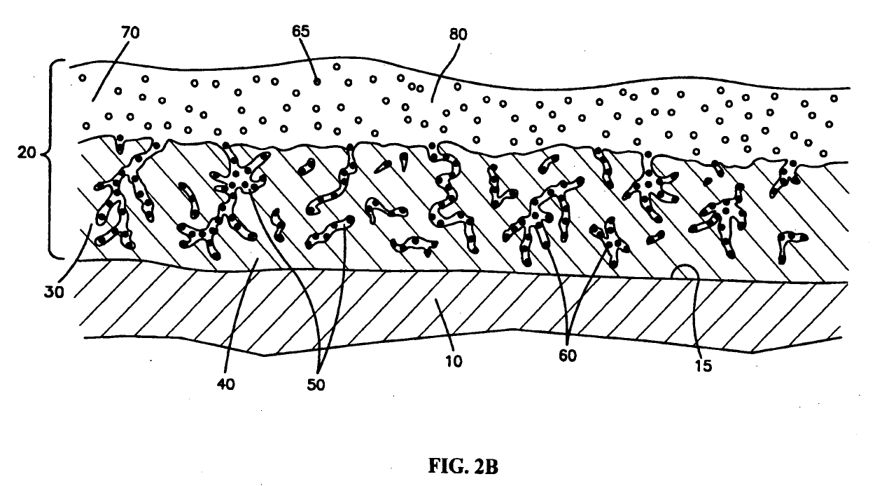

FIG. 2A shows an example of another embodiment. In this figure, the substrate 10 of the medical device, which can be a stent, has a surface 15 that is coated with coating 20. This coating 20 comprises a first coating composition 30 disposed on at least a portion of the surface 15. The first coating composition 30 comprises a non-releasable metal oxide 40 having a plurality of pores 50 therein. A first therapeutic agent 60 is disposed in at least some of the pores 50 of the non-releasable metal oxide 40. A second coating composition 70 is disposed on at least a portion of the first coating composition 30. The second coating composition 70 comprises a releasable metal oxide 80. In this embodiment, the releasable metal oxide is released when the medical device is implanted.

FIG. 2B shows a cross-sectional view of an example of a coated medical device that is similar to the one shown in FIG. 2A. However, the example shown in FIG. 2B, the second coating composition 70 includes a second therapeutic agent 65. The first and second therapeutic agents 60, 65 can be the same or different. In some embodiments, before it is applied to the medical device, the releasable metal oxide can comprise the therapeutic agent.

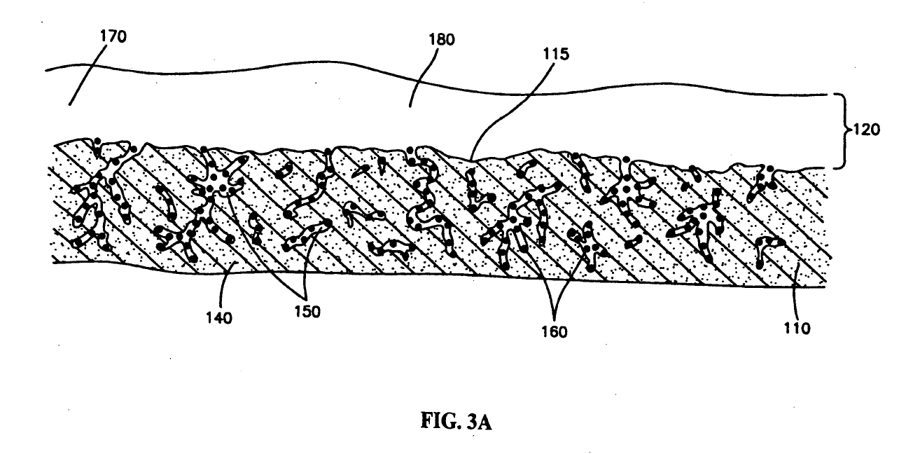

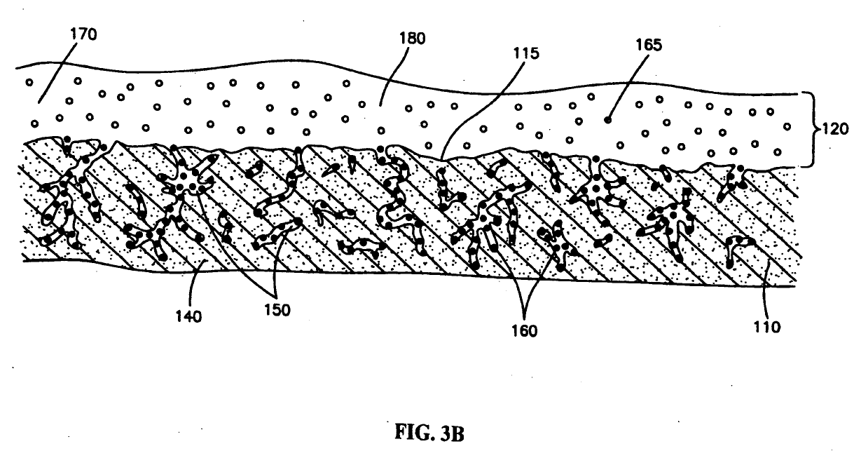

In another embodiment, the medical devices described herein comprise a substrate having a surface. The substrate is comprised of a non-releasable metal oxide having a plurality of pores therein. A coating is disposed on at least a portion of the surface of the substrate. The coating is free of any synthetic polymer, or in certain instances is free of any polymer, that is not part of any releasable metal oxide. FIG. 3A shows an example of such an embodiment. In this figure, the substrate 110 of the medical device, which can be a stent, has a surface 115 that is coated with coating 120. The substrate 110 comprises a non-releasable metal oxide 140 having a plurality of pores 150 therein. A first therapeutic agent 160 is disposed in at least some of the pores 150 of the non-releasable metal oxide 140. A coating 120 comprising a first coating composition 170 is disposed on at least a portion of the surface 115. The first coating composition 170 comprises a releasable metal oxide 180. When implanted in a patient, the releasable metal oxide is released from the medical device.

FIG. 3B shows a cross-sectional view of an example of a coated medical device that is similar to the one shown in FIG. 3A. However, in the example shown in FIG. 3B the first coating composition 170 includes a second therapeutic agent 165. The first and second therapeutic agents 160, 165 can be the same or different. In some embodiments, before it is applied to the medical device, the releasable metal oxide of the first coating composition can comprise the therapeutic agent.

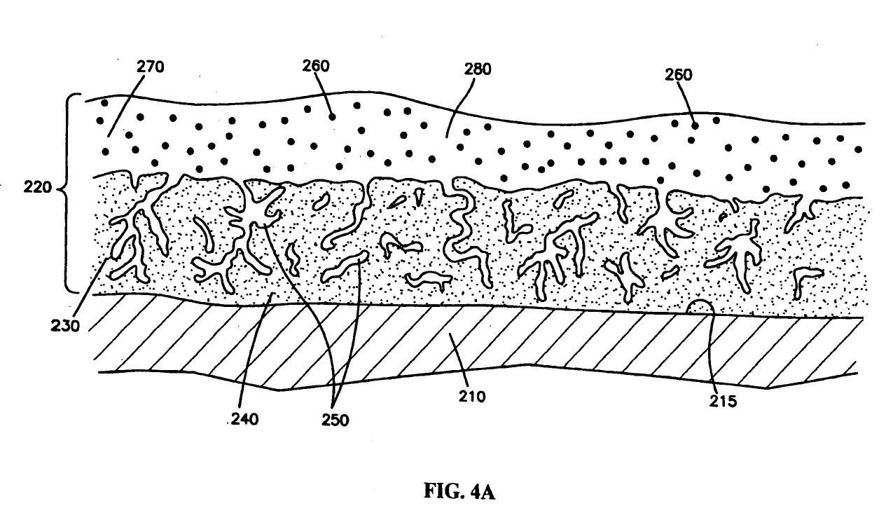

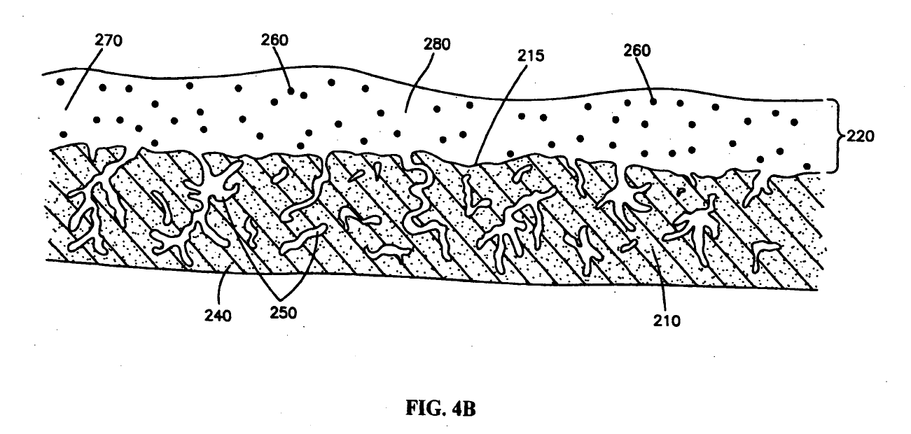

FIG. 4A shows a cross-sectional view of another embodiment of a coated medical device that is similar to the one shown in FIG. 2B. In this embodiment, a therapeutic agent is not disposed in the pores of the non-releasable metal oxide before a second coating composition comprising a releasable metal oxide and a therapeutic agent is disposed on the first coating composition. Specifically, as shown in FIG. 4A the substrate 210 of the medical device, which can be a stent, has a surface 215 that is coated with coating 220. This coating 220 comprises a first coating composition 230 disposed on at least a portion of the surface 215. The first coating composition 230 comprises a non-releasable metal oxide 240 having a plurality of pores 250 therein. A second coating composition 270 is disposed on at least a portion of the first coating composition 230. The second coating composition 270 comprises a releasable metal oxide 280 and a first therapeutic agent 260. In this embodiment, no therapeutic agent is disposed in the pores 250 of the non-releasable metal oxide 240 before the second coating composition 270 is disposed on the first coating composition 230. In some instances, a portion of the therapeutic agent 260 of the second coating composition 270 may become disposed in the pores 250. In some embodiments, before it is applied to the medical device, the releasable metal oxide of the second coating composition can comprise the therapeutic agent.

FIG. 4B shows a cross-sectional view of another embodiment of a coated medical device that is similar to the one shown in FIG. 3B. In this embodiment, a therapeutic agent is not disposed in the pores of the non-releasable metal oxide before a coating composition comprising a releasable metal oxide and a therapeutic agent is disposed on the surface of the medical device that comprises the non-releasable metal oxide. In particular, as shown in FIG. 4B the substrate 210 of the medical device, which can be a stent, has a surface 215 upon which a coating 220 is disposed. The substrate 210 comprises a non-releasable metal oxide 240 having a plurality of pores 250 therein. The coating 220 comprises a coating composition 270 comprising a releasable metal oxide 280 and first therapeutic agent 260. In this embodiment, no therapeutic agent is disposed in the pores 250 of the non-releasable metal oxide 240 before the coating composition 270 is disposed on the surface 215. In some instances, a portion of the therapeutic agent 260 of the coating composition 270 may become disposed in the pores 250. In some embodiments, before it is applied to the medical device, the releasable metal oxide of the coating composition can comprise the therapeutic agent.

It should be noted that while the coatings in the above described embodiments are shown as comprising one or two coating compositions, the coatings may comprise more than two coating compositions.

The pores in the non-releasable and releasable metal oxides can have various sizes. For example, at least some of the pores can have diameters or widths that range from about 1 nm to about 100 μm, about 1000 nm to about 1000 μm about 10 nm to about 10 μm, about 100 nm to about 10 μm, about 10 nm to about 1 μm, or about 100 nm to about 1 μm. In certain embodiments, the diameter or width of the pores in the coating composition is about 1 nm, about 10 nm, about 100 nm, about 1 μm, about 10 μm, about 100 μm. In some embodiments, the diameter or width of the pores of the coating composition is less than 1 nm, less than 10 nm, less than 100 nm, less than 1 μm, less than 10 μm, or less than 100 μm.

As shown in the above figures, the first and second coating compositions are generally in the form of layers. In other embodiments, the compositions need not be in the form of layers. If the coating compositions are in the form of layers, the layers can have a thickness of about 1 nm to about 1000 μm, about 10 nm to about 100 μm, about 1 nm to about 10 μm, or about 1 μm to about 100 μm.

The medical devices described herein are discussed in more detail in Section 5.1 infra. Methods of preparing the medical device described herein are discussed in Section 5.2, infra. For clarity of disclosure, and not by way of limitation, the detailed description is divided into the subsections which follow.

### 5.1 THE MEDICAL DEVICE

### 5.1.1 Types of Medical Devices

The medical devices described herein can be implanted or inserted into the body of a patient. Suitable medical devices include, but are not limited to, stents, surgical staples, catheters, such as balloon catheters, central venous catheters, and arterial catheters, guidewires, cannulas, cardiac pacemaker leads or lead tips, cardiac defibrillator leads or lead tips, implantable vascular access ports, blood storage bags, blood tubing, vascular or other grafts, intra aortic balloon pumps, heart valves, cardiovascular sutures, total artificial hearts and ventricular assist pumps, bone implants, and extra corporeal devices such as blood oxygenators, blood filters, septal defect devices, hemodialysis units, hemoperfusion units and plasmapheresis units.

Suitable medical devices include, but are not limited to, those that have a tubular or cylindrical like portion. For example, the tubular portion of the medical device need not be completely cylindrical. The cross-section of the tubular portion can be any shape, such as rectangle, a triangle, etc., not just a circle. Such devices include, but are not limited to, stents, balloon catheters, and grafts. A bifurcated stent is also included among the medical devices which can be fabricated by the methods described herein.

In addition, the tubular portion of the medical device may be a sidewall that may comprise a plurality of struts defining a plurality of openings. The sidewall defines a lumen. The struts may be arranged in any suitable configuration. Also, the struts do not all have to have the same shape or geometric configuration. When the medical device is a stent comprising a plurality of struts, the surface is located on the struts. Each individual strut has an outer surface adapted for exposure to the body tissue of the patient, an inner surface, and at least one side surface between the outer surface and the inner surface.

Medical devices that are particularly suitable include any kind of stent for medical purposes which is known to the skilled artisan. Preferably, the stents are intravascular stents that are designed for permanent implantation in a blood vessel of a patient. In certain embodiments, the stent comprises an open lattice sidewall stent structure, such as a coronary stent. Other suitable stents include, for example, vascular stents such as self-expanding stents and balloon expandable stents. Examples of useful self-expanding stents are illustrated in U.S. Pat. Nos. 4,655,771 and 4,954,126 issued to Wallsten and 5,061,275 issued to Wallsten et al. Examples of appropriate balloon-expandable stents are shown in U.S. Pat. No. 5,449,373 issued to Pinchasik et al.

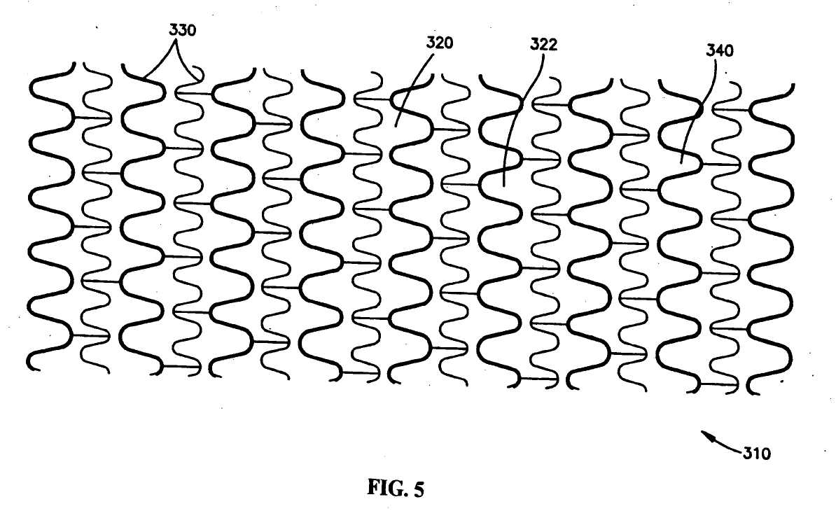

FIG. 5 shows an example of a medical device that is suitable for use in the embodiments described herein. This figure shows a peripheral view of an implantable intravascular stent 310. As shown in FIG. 5, the intravascular stent 310 is generally cylindrical in shape. Stent 310 includes a sidewall 320 which comprises a plurality of struts 330 and at least one opening 340 in the sidewall 320. Generally, the opening 340 is disposed between adjacent struts 330. Also, the sidewall 320 may have a first sidewall surface 322 and an opposing second sidewall surface, which is not shown in FIG. 5. The first sidewall surface 322 can be an outer or abluminal sidewall surface, which faces a body lumen wall when the stent is implanted, or an inner or luminal sidewall surface, which faces away from the body lumen surface. Likewise, the second sidewall surface can be an abluminal sidewall surface or a luminal sidewall surface.

When the coatings described herein are applied to a stent having openings in the stent sidewall structure, in certain embodiments, it is preferable that the coatings conform to the surface of the stent so that the openings in the sidewall stent structure are preserved, e.g. the openings are not entirely or partially occluded with coating material.

The framework of suitable stents may be formed through various methods as known in the art. The framework may be welded, molded, laser cut, electro-formed, or consist of filaments or fibers which are wound or braided together in order to form a continuous structure.

Suitable substrates of the medical device (e.g., stents) may be fabricated from a metallic material, ceramic material, or polymeric material or a combination thereof (see Sections 5.1.1.1 to 5.1.1.3 infra.). Preferably, the materials are biocompatible. The material may be porous or non-porous, and the porous structural elements can be microporous or nanoporous.

### 5.1.1.1. Metallic Materials for Device Formation

In certain embodiments, the medical devices described herein comprise a substrate which is metallic. Suitable metallic materials useful for making the substrate include, but are not limited to, metals and alloys based on titanium (such as nitinol, nickel titanium alloys, thermo memory alloy materials), stainless steel, gold, platinum, iridium, molybdenum, niobium, palladium, chromium, tantalum, nickel chrome, or certain cobalt alloys including cobalt chromium nickel alloys such as Elgiloy® and Phynox®, or a combination thereof. Other metallic materials that can be used to make the medical device include clad composite filaments, such as those disclosed in WO 94/16646.

Preferably, the metal or metal oxide region comprises a radiopaque material. Including a radiopaque material may be desired so that the medical device is visible under X-ray or fluoroscopy. Suitable materials that are radiopaque include, but are not limited to, gold, tantalum, platinum, bismuth, iridium, zirconium, iodine, titanium, barium, silver, tin, alloys of these metals, or a combination thereof.

Furthermore, although certain embodiments described herein can be practiced by using a single type of metal to form the substrate, various combinations of metals can also be employed. The appropriate mixture of metals can be coordinated to produce desired effects when incorporated into a substrate.

### 5.1.1.2. Ceramic Materials for Device Formation

In certain embodiments, the medical device described herein comprises a substrate which is ceramic. Suitable ceramic materials used for making the substrate include, but are not limited to, oxides, carbides, or nitrides of the transition elements such as titanium oxides, platinum oxide, tantalum oxide, hafnium oxides, iridium oxides, chromium oxides, niobium oxide, tungsten oxide, rhodium oxide, aluminum oxides, zirconium oxides, or a combination thereof. Silicon based materials, such as silica, may also be used.

Furthermore, although certain embodiments described herein can be practiced by using a single type of ceramic to form the substrate, various combinations of ceramics can also be employed. The appropriate mixture of ceramics can be coordinated to produce desired effects when incorporated into a substrate.

### 5.1.1.3. Polymeric Materials for Device Formation

In certain embodiments, the medical devices described herein comprise a substrate which is polymeric. In other embodiments, the material can be non-polymeric. The polymer(s) useful for forming the components of the medical devices should be ones that are biocompatible and avoid irritation to body tissue. The polymers can be biostable or bioabsorbable. Suitable polymeric materials useful for making the substrate include, but are not limited to, isobutylene-based polymers; polystyrene-based polymers such as styrene isobutylene styrene co-polymers; polyacrylates and polyacrylate derivatives such as polycyanoacrylates, ethylene glycol I dimethacrylate, poly(methyl methacrylate) and, poly(2-hydroxyethyl methacrylate); vinyl acetate-based polymers and copolymers such as ethylene vinyl-acetate; polyurethane and its copolymers; silicone and its copolymers; polyethylene terephtalate; thermoplastic elastomers; polyvinyl chloride; polyolefins; cellulosics; polyamides; polyesters such as Dacron polyester and poly(ortho ester); polysulfones; polytetrafluorethylenes; polycarbonates such as polyiminocarbonates; acrylonitrile butadiene styrene copolymers; acrylics; polylactic acid; polyglycolic acid; poly(glycolide-lactide) co-polymers; polycaprolactone; polypropylene; polyalkylene oxalates; polysiloxanes such as poly(dimethyl siloxane); nylons; polyphosphazenes; poly(amino acids); poly(HEMA); polyhydroxyalkanoates; polyhydroxybutyrate; polydioxanone; poly(y-ethyl glutamate); polyanhydrides; polyetheroxides; polyvinyl alcohols; polylactic acid-polyethylene oxide copolymers; collagens; chitins; Teflon; alginate; dextran; cotton; and combinations and derivatized versions thereof, (i.e., polymers which have been modified to include, for example, attachment sites or cross-linking groups, e.g., arginine-glycine-aspartic acid RGD, in which the polymers retain their structural integrity while allowing for attachment of cells and molecules, such as proteins and/or nucleic acids).

The polymers may be dried to increase their mechanical strength. The polymers may then be used as the base material to form a whole or part of the substrate. Furthermore, although certain embodiments can be practiced by using a single type of polymer to form the substrate, various combinations of polymers can also be employed. The appropriate mixture of polymers can be coordinated to produce desired effects when incorporated into a substrate.

### 5.1.2 Therapeutic Agents

The term “therapeutic agent” as used herein encompasses drugs, genetic materials, and biological materials and can be used interchangeably with “biologically active material.” The term “genetic materials” means DNA or RNA, including, without limitation, DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other suitable therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, pimecrolimus, sirolimus, zotarolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives, paclitaxel as well as its derivatives, analogs or paclitaxel bound to proteins, e.g. Abraxane™;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, daunomycin, mitocycin;
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds;
macrolides such as sirolimus (rapamycin) or everolimus; and
AGE-breakers including alagebrium chloride (ALT-711).

Other therapeutic agents include nitroglycerin, nitrous oxides, nitric oxides, antibiotics, aspirins, digitalis, estrogen, estradiol and glycosides. Preferred therapeutic agents include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the embodiments described herein include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, and 2′-O-ester with N-(dimethylaminoethyl)glutamide hydrochloride salt.

Other preferred therapeutic agents include tacrolimus; halofuginone; inhibitors of HSP90 heat shock proteins such as geldanamycin; microtubule stabilizing agents such as epothilone D; phosphodiesterase inhibitors such as cliostazole; Barket inhibitors; phospholamban inhibitors; and Serca 2 gene/proteins. In yet another preferred embodiment, the therapeutic agent is an antibiotic such as erythromycin, amphotericin, rapamycin, adriamycin, etc.

In preferred embodiments, the therapeutic agent comprises daunomycin, mitocycin, dexamethasone, everolimus, tacrolimus, zotarolimus, heparin, aspirin, warfarin, ticlopidine, salsalate, diflunisal, ibuprofen, ketoprofen, nabumetone, prioxicam, naproxen, diclofenac, indomethacin, sulindac, tolmetin, etodolac, ketorolac, oxaprozin, celcoxib, alagebrium chloride or a combination thereof.

The therapeutic agents can be synthesized by methods well known to one skilled in the art. Alternatively, the therapeutic agents can be purchased from chemical and pharmaceutical companies.

### 5.1.3. Non-Releasable and Releasable Metal Oxides

Suitable metal oxides of the non-releasable metal oxides and releasable metal oxides include but are not limited to, metal oxides that contain one or more of the following metals: titanium, scandium, iron, tantalum, nickel, cobalt, chromium, manganese, platinum, iridium, niobium, vanadium, zirconium, tungsten, rhodium, ruthenium, gold, copper, zinc, yttrium, molybdenum, technetium, palladium, cadmium, hafnium, rhenium and combinations thereof. In certain embodiments, preferred metals include without limitation, gold, tantalum, platinum, titanium, iridium or a combination thereof.

Examples of suitable metal oxides include without limitation: platinum oxides, tantalum oxides, titanium oxides, zinc oxides, iron oxides, magnesium oxides, aluminum oxides, iridium oxides, niobium oxides, zirconium oxides, tungsten oxides, rhodium oxides, ruthenium oxides, alumina, zirconia, silicone oxides such as silica based glasses and silicon dioxide, or combinations thereof.

The metal oxides can also be mixed metal oxides such any of tin/tetravalent tin oxide, tin/divalent tin oxide, tin/indium oxide, tin/antimony oxide, tin/zinc oxide, tin/titanium oxide, tin/vanadium oxide, tin/chromium oxide, tin/manganese oxide, tin/iron oxide, tin/cobalt oxide, tin/nickel oxide, tin/zirconium oxide, tin/molybdenum oxide, tin/palladium oxide, tin/iridium oxide, tin/magnesium oxide, titanium/tetravalent titanium oxide, titanium divalent titanium oxide, titanium/indium oxide, titanium/antimony oxide, titanium/zinc oxide, titanium/tin oxide, titanium/vanadium oxide, titanium/chromium oxide, titanium/manganese oxide, titanium/iron oxide, titanium/cobalt oxide, titanium/nickel oxide, titanium/zirconium oxide, titanium/molybdenum oxide, titanium/palladium oxide, titanium/iridium oxide, or titanium/magnesium oxide.

In certain embodiments, the releasable metal oxide is an adduct of at least one metal oxide and an organic group. PCT Publication WO 2005/049520, which is incorporated by reference in its entirety for all purposes, describes examples of releasable metal oxides comprising such compounds. In some embodiments, the adduct can comprise more than one type of organic group. The organic group can be attached to a metal or to another organic group of the adduct. For example, the adduct can comprise a metal oxide, a first organic group and a second organic group that is attached to the first organic group or the metal oxide. In some embodiments, the second organic group is a therapeutic agent. Such therapeutic agents can be attached to the adduct by, for example, hydrogen bonding. The organic group can, for example, be an acetate, such as a fluoroacetate; a phosphate, a polyethylene glycol, a polymer, a therapeutic agent or any chemical moiety that can be bonded to the adduct.

### 5.2. METHODS OF MAKING THE COATINGS

Provided herein are methods of making the medical devices described above. In one embodiment, the method of making the implantable coated stent comprises providing an implantable stent that has substrate, which has a surface. A coating that is free of any synthetic polymer or any polymer that is not part of any releasable metal oxide is formed on at least a portion of the surface by applying a first coating composition onto at least a portion of the surface. The first coating composition comprises a releasable metal oxide. In some embodiments, the first coating composition includes a therapeutic agent. Also, the method can further include applying a second coating composition that comprises a second releasable metal oxide on the surface before the first coating composition is applied. The second coating composition is therefore disposed between the surface and the first coating composition. The second coating composition can contain a therapeutic agent or be free of any therapeutic agent.

For example, the methods described herein include a method of making an implantable coated stent by providing an implantable stent that includes a substrate having a surface and forming a coating that is free of any synthetic polymer that is not part of any releasable metal oxide on at least a portion of the surface. The coating is formed by applying a first coating composition to at least a portion of the surface and applying a second coating composition onto at least a portion of the first coating composition. The first coating composition includes a releasable metal oxide having an adduct of a titanium oxide and an acetate. In this embodiment, the first coating composition is free of a therapeutic agent when applied to the surface. The second coating composition includes paclitaxel and a releasable metal oxide having an adduct of a titanium oxide and an acetate.

In another embodiment, the method of making the coated medical device comprises providing an implantable medical device, such as a stent, which comprises a substrate having a surface. A coating, which is free of any synthetic polymer or free of any polymer that is not part of any releasable metal oxide, is formed on at least a portion of the surface. The coating is formed by forming a first coating composition onto at least a portion of the surface. The first coating composition comprises a non-releasable metal oxide. A plurality of pores is present in the non-releasable metal oxide. In some embodiments, a first therapeutic agent may be disposed in at least some of the pores of the non-releasable metal oxide. A second coating composition comprising a releasable metal oxide is applied onto at least a portion of the first coating composition. This second coating composition may also include a therapeutic agent. In some embodiments, the non-releasable metal oxide of the first coating composition is formed by applying a composition comprising a releasable metal oxide to the surface. The releasable metal oxide is exposed to a heat source to form the non-releasable metal oxide.

In another embodiment, the method for making an implantable coated medical device comprises forming an implantable medical device, such as a stent, that comprises a substrate having a surface. The substrate comprises a non-releasable metal oxide and a plurality of pores therein. In some embodiments, a first therapeutic agent may be disposed in at least some of the pores. A coating, which is free of any synthetic polymer or free of any polymer that is not part of any releasable metal oxide, is formed on the surface of the substrate. The coating is perpared by applying a coating composition onto at least a portion of the surface, wherein the coating composition comprises a releasable metal oxide, and in some instances also a therapeutic agent.

For example, the methods described herein include a method of making an implantable coated stent comprising forming an implantable stent that has a substrate and a surface and forming a coating on the surface of the substrate by applying a coating composition onto at least a portion of the surface. The substrate is a non-releasable metal oxide having a plurality of pores therein and the coating composition includes paclitaxel and a releasable metal oxide comprising an adduct of a titanium oxide and an acetate. Additionally, the coating is free of any synthetic polymer that is not part of any releasable metal oxide.

In yet another embodiment, the method of making an implantable coated stent comprises providing an implantable stent comprising a substrate having a surface and forming a coating that is free of any synthetic polymer that is not part of any releasable metal oxide on at least a portion of the surface. Forming the coating includes the steps of applying a solution or suspension of a releasable metal oxide onto at least a portion of the surface, exposing the stent to a heat source to form a first coating composition on at least a portion of the surface and applying a second coating composition onto at least a portion of the first coating composition. The solution or suspension of the metal oxide can include an adduct of a titanium oxide and an acetate. The first coating composition is a non-releasable metal oxide comprising the titanium oxide and a plurality of pores in the non-releasable metal oxide. Additionally, the second coating composition includes paclitaxel and a releasable metal oxide comprising an adduct of a titanium oxide and an acetate.

### 5.2.1. Preparing a Porous Substrate

The pores of the substrate can be created by any method known to one skilled in the art including, but not limited to, sintering, co-deposition, micro-roughing, laser ablation, drilling, chemical etching or a combination thereof. For example, the porous structure can be made by a deposition process such as sputtering with adjustments to the deposition condition, by micro-roughening using reactive plasmas, by ion bombardment, electrolyte etching, or a combination thereof. Other methods include, but are not limited to, alloy plating, physical vapor deposition, chemical vapor deposition, sintering, or a combination thereof.

Additionally, the pores can be formed by removing a secondary material such as a spacer group from the non-releasable metal oxide used to form the substrate. In particular, the substrate is formed from a composition containing the non-releasable metal oxide and the secondary material. The secondary material is then removed. Techniques for removing a secondary material include, but are not limited to, dealloying or anodization processes, or by baking or heating to remove the secondary material. The secondary material can be any material so long as it can be removed from the non-releasable metal oxide. For example, the secondary material can be more electrochemically active than the non-releasable metal oxide. Also, the spacer group or secondary material can be an organic group that is bonded to the releasable metal oxide such as those described above.

In embodiments where a therapeutic agent is disposed in pores, the therapeutic agent can be dispersed in the pores of the substrate by any method known to one skilled in the art including, but not limited to, dipping, spray coating, spin coating, plasma deposition, condensation, electrochemically, electrostatically, evaporation, plasma vapor deposition, cathodic arc deposition, sputtering, ion implantation, use of a fluidized bed, or a combination thereof. Methods suitable for dispersing the therapeutic agent into the pores of the substrate preferably do not alter or adversely impact the therapeutic properties of the therapeutic agent. To facilitate the disposition of the therapeutic agent into the pores, the therapeutic agent can be placed into a solution or suspension containing a solvent or carrier. For instance, a solution containing the therapeutic agent can be formed and the medical device can be dipped into the solution to allow the therapeutic agent to be disposed in the pores.

### 5.2.2. Application of Coating Compositions

The coating compositions are preferably formed by applying a solution or suspension that contains the desired constituents. For instance, to form a coating composition that contains a releasable metal oxide, such oxide can be dissolved or suspended in a solvent. Suitable solvents include without limitation methanol, water, acetone, ethanone, butanone, and THF. The solution or suspension can also include a therapeutic agent.

The solutions or suspensions can be applied to at least a portion of a surface of a substrate or another coating composition by any method known to one skilled in the art, including, but not limited to, dipping, spraying, such as by conventional nozzle or ultrasonic nozzle, laminating, pressing, brushing, swabbing, dipping, rolling, electrostatic deposition, painting, electroplating, evaporation, plasma-vapor deposition, a batch process such as air suspension, pan coating or ultrasonic mist spraying, cathodic-arc deposition, sputtering, ion implantation, electrostatically, electroplating, electrochemically, and chemical methods of immobilization of bio-molecules to surfaces, or a combination thereof. Preferably, the coating composition is applied by spraying, dipping, laminating, pressing, or a combination thereof.

### 5.2.3. Preparing a Coating Composition Comprising a Non-Releasable Metal Oxide Having a Plurality of Pores Therein

As discussed above, the coating composition can comprise a non-releasable metal oxide having a plurality of pores therein. The pores in the non-releasable metal oxides can be created by any method known to one skilled in the art including, but not limited to, the ones discussed above in connection with the formation of pores in the non-releasable metal oxides used to form the substrate. For example, the pores can be formed by removing a secondary material, such as a spacer group, from the non-releasable metal oxide in the coating compositions. In particular, the coating composition includes a metal oxide and a secondary material. After the coating composition is applied to the substrate or another coating composition, the secondary material is removed to create pores in the metal oxide. In other embodiments, the pores can be formed when the metal oxide is applied to the surface of the medical device or another coating composition.

In one embodiment, the non-releasable metal oxide having a plurality of pores is formed by using a solution or suspension of a releasable metal oxide. The solution or suspension is applied onto the surface or a coating composition and then exposed to a heat or energy source to form the non-releasable metal oxide with the plurality of pores. In some embodiments, the solution or suspension applied to the surface or coating composition is heated up to about 900° C., but lower temperatures can also be used depending on the degree of annealing or porosity or crystalline phase required or the type of spacer group being removed. Also, in some instances, where the releasable metal oxide is an adduct of a metal oxide and an organic group, the exposure to the heat or energy source is sufficient to remove the organic group and results in the formation of the pores. By varying the type of organic group used, the sizes of the pores can be varied. Furthermore, therapeutic agents can be disposed in the pores by the methods discussed above in connection with the disposition of therapeutic agents in the pores of the substrate.

The following examples are for purposes of illustration and not for purposes of limitation.

### 5.3 EXAMPLES

### Example 1

Nine (9) stainless steel coupons were prepared as described in Table 1 below:

TABLE 1

| Coupon # | Description |
|---|---|
| 1 | Three coatings of titanium (IV) oxide on a coupon were |
| | prepared by using titanium (IV) oxide trifluoroacetate in |
| | butanone (100 g/L, 0.3 cm³ per coating) and spin coating |
| | (1st spin 500 rpm, 2nd spin 3000 rpm). Each coating was |
| | annealed at 800° C. for two hours. This sample did not |
| | include any therapeutic agent and was used as a control |
| | sample. |
| 2 | Three coatings of titanium (IV) oxide on a coupon were |
| | prepared by using titanium (IV) oxide trifluoroacetate in |
| | butanone (100 g/L, 0.3 cm³ per coating) and spin coating |
| | (1st spin 500 rpm, 2nd spin 3000 rpm). Each coating was |
| | annealed at 800° C. for two hours. This sample was soaked |
| | in a 1% solution of paclitaxel in ethanol for sixty hours at |
| | room temperature and then dried in the open air. |
| 3 | Three coatings of titanium (IV) oxide on a coupon were |
| | prepared by using titanium (IV) oxide trifluoroacetate in |
| | butanone (100 g/L, 0.3 cm³ per coating) and spin coating |
| | (1st spin 500 rpm, 2nd spin 3000 rpm). Each coating was |
| | annealed at 800° C. for two hours. This sample was soaked |
| | in a 1% solution of paclitaxel in ethanol for sixty hours at |
| | room temperature and then dried in the open air. |
| | Subsequently, the sample was coated with a 50:50 (w/w) |
| | solution of titanium (IV) oxide trifluoroacetate and |
| | paclitaxel in butanone (0.5 g TiO₂/TFA, 0.5 g paclitaxel, |
| | 5 cm³ butanone). |
| 4 | Three coatings of titanium (IV) oxide on a coupon were |
| | prepared by using titanium (IV) oxide trifluoroacetate in |
| | butanone (100 g/L, 0.3 cm³ per coating) and spin coating |
| | (1st spin 500 rpm, 2nd spin 3000 rpm). Each coating was |
| | annealed at 800° C. for two hours. This sample was soaked |
| | in a 1% solution of paclitaxel in ethanol for sixty hours at |
| | room temperature and then dried in the open air. |
| | Subsequently, the sample was coated with titanium (IV) |
| | oxide using titanium (IV) oxide trifluoroacetate in butanone |
| | (100 g/L, 0.3 cm³ solution) and spin coating (1st spin |
| | 500 rpm, 2nd spin 3000 rpm), and then heating to 70° C. for |
| | two hours. |
| 5 | Three coatings of titanium (IV) oxide on a coupon were |
| | prepared by using titanium (IV) oxide trifluoroacetate in |
| | butanone (100 g/L, 0.3 cm³ per coating) and spin coating |
| | (1st spin 500 rpm, 2nd spin 3000 rpm). Each coating was |
| | annealed at 800° C. for two hours. This sample was not |
| | soaked in 1% paclitaxel in ethanol but was coated directly |
| | with a 50:50 (w/w) solution of paclitaxel:TiO₂/TFA in |
| | butanone (0.5 g paclitaxel, 0.5 g TiO₂/TFA in 5 cm³ |
| | butanone, 0.3 cm³ solution) using spin coating (1st spin |
| | 500 rpm, 2nd spin 3000 rpm). This sample was then heated |
| | to 70° C. for two hours. |
| 6 | Uncoated 18/8 stainless steel coupon was soaked in a 1% |
| | solution of paclitaxel in ethanol for sixty hours at room |
| | temperature and then dried in the open air and was used as |
| | a control sample. |
| 7 | Three coatings of titanium (IV) oxide on a coupon were |
| | prepared by using titanium (IV) oxide trifluoroacetate in |
| | butanone (100 g/L, 0.3 cm³ per coating) and spin coating |
| | (1st spin 500 rpm, 2nd spin 3000 rpm). Each coating was |
| | annealed at 270° C. for two hours. This sample was soaked |
| | in a 1% solution of paclitaxel in ethanol for twenty four |
| | hours at room temperature and then dried in the open air. |
| 8 | Three coatings of titanium (IV) oxide on a coupon were |
| | prepared by using titanium (IV) oxide trifluoroacetate in |
| | butanone (100 g/L, 0.3 cm³ per coating) and spin coating |
| | (1st spin 500 rpm, 2nd spin 3000 rpm). Each coating was |
| | annealed at 270° C. for two hours. This sample was not |
| | soaked in 1% paclitaxel in ethanol but was coated directly |
| | with a 50:50 (w/w) solution of paclitaxel:TiO₂/TFA in |
| | butanone (0.5 g paclitaxel, 0.5 g TiO₂/TFA in 5 cm³ |
| | butanone, 0.3 cm³ solution) using spin coating (1st spin |
| | 500 rpm, 2nd spin 3000 rpm). This sample was then heated |
| | to 70° C. for two hours. |
| 9 | Three coatings of titanium (IV) oxide on a coupon were |
| | prepared by using titanium (IV) oxide trifluoroacetate in |
| | butanone (100 g/L, 0.3 cm³ per coating) and spin coating |
| | (1st spin 500 rpm, 2nd spin 3000 rpm). Each coating was |
| | annealed at 270° C. for two hours. This sample did not |
| | include any therapeutic agent and was used as a control |
| | sample. |

The titanium (IV) trifluoroacetate was prepared according to the methods described in PCT Publication No. WO2005/049520. The 50:50 (w/w) solution of paclitaxel:TiO₂/TFA was prepared by reacting the soluble TiO₂/TFA material with paclitaxel in ethanol in a 1:1 ratio and the surface derivatized titanium (IV) oxide paclitaxel material was isolated in a solid state and re-dissolved in butanone (10% solids/90% butanone). Other solvents could potentially be used as well.

The coated coupons were placed in a buffered solution of saline with 0.05% (w/v) Tween 20 at a pH of 7.4. The amount of paclitaxel released from each coupon into the buffered solution over time was measured using HPLC detection by UV. Table 2 below sets forth the amount of paclitaxel released from each coupon over time.

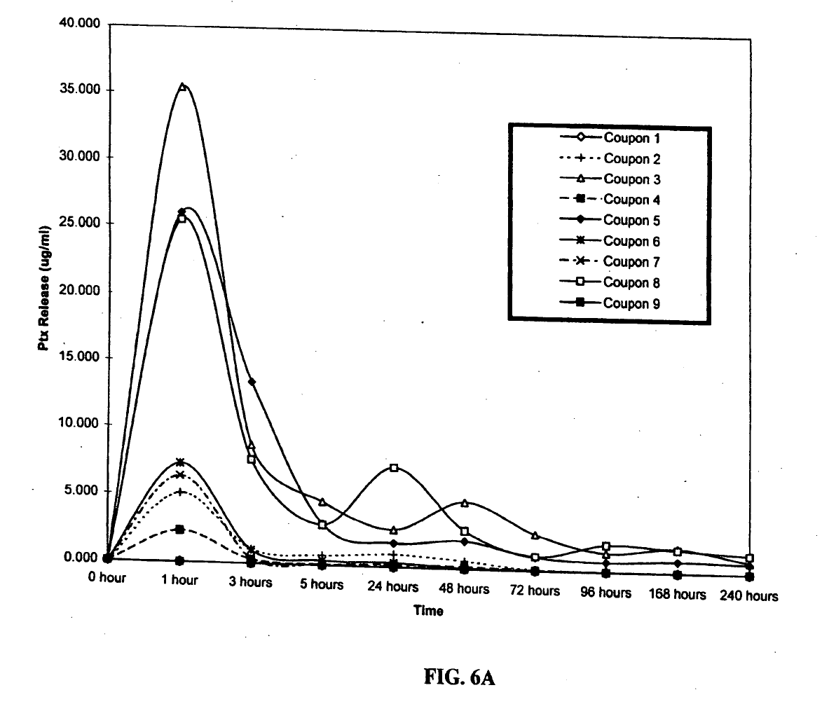

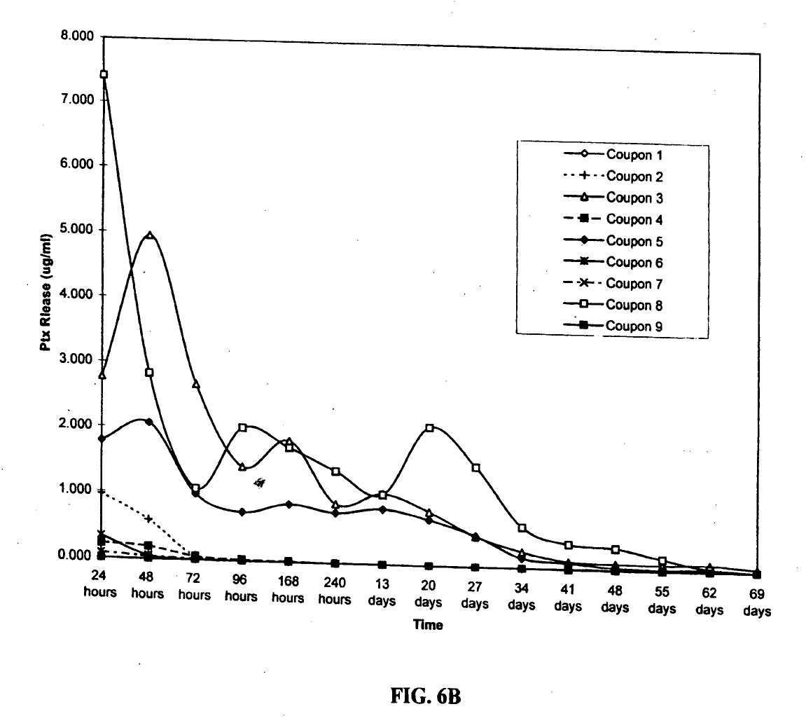

FIGS. 6A-6B are a graphical representation of the amount of paclitaxel released from each of the coupons over time.

TABLE 2

| | Coupon | Coupon | Coupon | Coupon | Coupon | Coupon | Coupon | Coupon | Coupon |
|---|---|---|---|---|---|---|---|---|---|
| | 1 | 2 | 3 | 4 | 5 | 6 | 7 | 8 | 9 |
| 0 | 0.000 | 0.000 | 0.000 | 0.000 | 0.000 | 0.000 | 0.000 | 0.000 | 0.000 |
| hours | | | | | | | | | |
| 1 | 0.000 | 5.109 | 35.419 | 2.315 | 26.023 | 7.344 | 6.404 | 25.513 | 0.000 |
| hours | | | | | | | | | |
| 3 | 0.000 | 1.066 | 8.790 | 0.313 | 13.509 | 0.992 | 0.383 | 7.715 | 0.000 |
| hours | | | | | | | | | |
| 5 | 0.000 | 0.759 | 4.707 | 0.073 | 3.064 | 0.355 | 0.056 | 2.970 | 0.000 |
| hours | | | | | | | | | |
| 24 | 0.000 | 0.963 | 2.764 | 0.219 | 1.781 | 0.325 | 0.076 | 7.402 | 0.000 |
| hours | | | | | | | | | |
| 48 | 0.000 | 0.585 | 4.946 | 0.180 | 2.064 | 0.045 | 0.035 | 2.825 | 0.000 |
| hours | | | | | | | | | |
| 72 | 0.000 | 0.053 | 2.671 | 0.048 | 0.985 | 0.000 | 0.012 | 1.069 | 0.000 |
| hours | | | | | | | | | |
| 96 | 0.000 | 0.000 | 1.420 | 0.016 | 0.727 | 0.000 | 0.000 | 2.019 | 0.000 |
| hours | | | | | | | | | |
| 168 | 0.000 | 0.000 | 1.837 | 0.009 | 0.867 | 0.000 | 0.000 | 1.729 | 0.000 |
| hours | | | | | | | | | |
| 240 | 0.000 | 0.000 | 0.892 | 0.000 | 0.757 | 0.000 | 0.000 | 1.392 | 0.000 |
| hours | | | | | | | | | |
| 13 | 0.000 | 0.000 | 1.063 | 0.000 | 0.833 | 0.000 | 0.000 | 1.052 | 0.000 |
| days | | | | | | | | | |
| 20 | 0.000 | 0.000 | 0.811 | 0.000 | 0.690 | 0.000 | 0.000 | 2.103 | 0.000 |
| days | | | | | | | | | |
| 27 | 0.000 | 0.000 | 0.458 | 0.000 | 0.468 | 0.000 | 0.000 | 1.513 | 0.000 |
| days | | | | | | | | | |
| 34 | 0.000 | 0.000 | 0.246 | 0.000 | 0.152 | 0.000 | 0.000 | 0.617 | 0.000 |
| days | | | | | | | | | |
| 41 | 0.000 | 0.000 | 0.115 | 0.000 | 0.098 | 0.000 | 0.000 | 0.375 | 0.000 |
| days | | | | | | | | | |
| 48 | 0.000 | 0.000 | 0.096 | 0.000 | 0.031 | 0.000 | 0.000 | 0.325 | 0.000 |
| days | | | | | | | | | |
| 55 | 0.000 | 0.000 | 0.087 | 0.000 | 0.014 | 0.000 | 0.000 | 0.178 | 0.000 |
| days | | | | | | | | | |
| 62 | 0.000 | 0.000 | 0.101 | 0.000 | 0.029 | 0.000 | 0.000 | 0.037 | 0.000 |
| days | | | | | | | | | |
| 69 | 0.000 | 0.000 | 0.048 | 0.000 | 0.000 | 0.000 | 0.000 | 0.000 | 0.000 |
| days | | | | | | | | | |

### Example 2

A stent with a coating can be prepared as follows. A composition of titanium (IV) oxide trifluoroacetate in butanone (e.g. 100 g/L, 0.3 cm³ per coating) can be spin coated (at for example speeds of about 500 rpm to about 3000 rpm). The stent with the composition disposed thereon is annealed at 800° C., or a lower temperature, for two hours. Afterwards, the stent can be soaked in a 1% solution of paclitaxel in ethanol for sixty hours at room temperature. The stent can then be dried in the open air. Subsequently, the stent can be coated with a 50:50 (w/w) solution of titanium (IV) oxide trifluoroacetate and paclitaxel in butanone (e.g. 0.5 g TiO₂/TFA, 0.5 g paclitaxel, 5 cm³ butanone) to form the coating.

### Example 3

A stent with a coating can be prepared as follows. A composition of titanium (IV) oxide trifluoroacetate in butanone (e.g. 100 g/L, 0.3 cm³ per coating) can be spin coated (at for example speeds of about 500 rpm to about 3000 rpm). The stent with the composition disposed thereon is annealed at 800° C., or a lower temperature, for two hours. The stent is then coated with a 50:50 (w/w) solution of paclitaxel:TiO₂/TFA in butanone (e.g. 0.5 g paclitaxel, 0.5 g TiO₂/TFA in 5 cm³ butanone, 0.3 cm³ solution) using spin coating (at for example speeds of about 500 rpm to about 300 rpm). The stent can then be heated to 70° C. for two hours to form the coating.

### Example 4

A stent with a coating can be prepared as follows. A composition of titanium (IV) oxide trifluoroacetate in butanone (e.g. 100 g/L, 0.3 cm³ per coating) can be spin coated (at for example speeds of about 500 rpm to about 300 rpm). The stent with the composition disposed thereon is annealed at 270° C. for two hours. The stent is then coated with a 50:50 (w/w) solution of paclitaxel:TiO₂/TFA in butanone (e.g. 0.5 g paclitaxel, 0.5 g TiO₂/TFA in 5 cm³ butanone, 0.3 cm³ solution) using spin coating (at for example speeds of about 500 rpm to about 3000 rpm). The stent can then be heated to 70° C. for two hours to form the coating. It should be noted that stents can be coated using a variety of techniques such as, but not limited to, roller coating, dip coating and spray coating. Also, other solvents with higher or lower boiling points can be used should the drying rate needs to be changed.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference in their entirety into the specification to the same extent as if each individual publication, patent or patent application was specifically and individually indicated to be incorporated herein by reference. Citation or discussion of a reference herein shall not be construed as an admission that such is prior art.

What is claimed is:

1. An implantable coated stent comprising:
(a) a substrate having a surface; and
(b) a coating disposed on at least a portion of the surface, wherein the coating comprises a first coating composition disposed on at least a portion of the surface, wherein the first coating composition comprises a first releasable metal oxide and a first therapeutic agent, and wherein the coating is free of any synthetic polymer that is not part of any releasable metal oxide.

2. The stent of claim 1, wherein the coating is free of any polymer that is not part of the releasable metal oxide.

3. The stent of claim 1, wherein the releasable metal oxide comprises an adduct of a metal oxide and an organic group.

4. The stent of claim 3, wherein the metal oxide of the adduct comprises a titanium oxide or an iridium oxide.

5. The stent of claim 3, wherein the organic group of the adduct comprises an acetate, a phosphate or a polyethylene glycol.

6. The stent of claim 3, wherein the metal oxide of the adduct comprises a titanium oxide and the organic group of the adduct comprises an acetate.

7. The stent of claim 3, wherein the adduct further comprises a second therapeutic agent that is hydrogen bonded to the metal oxide or the organic group.

8. The stent of claim 1, wherein the first therapeutic agent comprises an anti-thrombogenic agent, anti-angiogenesis agent, anti-proliferative agent, antibiotic, anti-restenosis agent, growth factor, immunosuppressant or radiochemical.

9. The stent of claim 1, wherein the first therapeutic agent comprises an anti-proliferative agent that inhibits smooth muscle cell proliferation.

10. The stent of claim 1, wherein the first therapeutic agent comprises paclitaxel.

11. The stent of claim 1, wherein the first therapeutic agent comprises sirolimus, tacrolimus, pimecrolimus, zotarolimus or everolimus.

12. The stent of claim 1 wherein the coating further comprises a second coating composition that comprises a second releasable metal oxide and that is disposed on the surface, wherein the second coating composition is disposed between the surface and the first coating composition.

13. The stent of claim 12, wherein the first and second releasable metal oxides are the same.

14. The stent of claim 12, wherein the second coating composition is free of a therapeutic agent when disposed on the surface.

15. The stent of claim 12, wherein the second coating composition comprises second a therapeutic agent.

16. The stent of claim 1, wherein the first coating composition is in the form of a layer having a thickness of about 1 micron to about 30 microns.

17. The stent of claim 1, wherein the substrate comprises a stent sidewall structure comprising a plurality of struts and openings therein.

18. The stent of claim 17, wherein the coating conforms to the stent sidewall in a manner such that the openings are preserved.

19. An implantable coated stent comprising:
(a) a substrate having a surface; and
(b) a coating disposed on at least a portion of the surface, wherein the coating comprises:
(i) a first coating composition disposed on at least a portion of the surface, wherein the first coating composition comprises paclitaxel and a first releasable metal oxide that comprises an adduct of a titanium oxide and an acetate; and
(ii) a second coating composition disposed between the surface and the first coating composition, wherein the second coating composition comprises the adduct and is free of a therapeutic agent when disposed on the surface, and
wherein the coating is free of any synthetic polymer that is not part of any releasable metal oxide.

20. An implantable coated stent comprising:
(a) a substrate having a surface; and
(b) a coating disposed on at least a portion of the surface, wherein the coating comprises:
(i) a first coating composition disposed on at least a portion of the surface, wherein the first coating composition comprises a non-releasable metal oxide having a plurality of pores therein; and
(ii) a second coating composition comprising a releasable metal oxide disposed on at least a portion of the first coating composition,
wherein the coating is free of any synthetic polymer that is not part of any releasable metal oxide.

21. The stent of claim 20, wherein the coating is free of any polymer that is not part of any releasable metal oxide.

22. The stent of claim 20, wherein the non-releasable metal oxide comprises a titanium oxide or an iridium oxide.

23. The stent of claim 20, wherein the releasable metal oxide comprises an adduct of a metal oxide and an organic group.

24. The stent of claim 23, wherein the metal oxide of the adduct comprises a titanium oxide or an iridium oxide.

25. The stent of claim 23, wherein the organic group of the adduct comprises an acetate, a phosphate or a polyethylene glycol.

26. The stent of claim 23, wherein the metal oxide of the adduct comprises a titanium oxide and the organic group of the adduct comprises an acetate.

27. The stent of claim 23, wherein the adduct further comprises a therapeutic agent that is hydrogen bonded to the metal oxide or the organic group.

28. The stent of claim 20 further comprising a therapeutic agent disposed in the pores of the non-releasable metal oxide.

29. The stent of claim 20, wherein the pores are free of any therapeutic agent before the second coating composition is disposed on the first coating composition.

30. The stent of claim 20, wherein the second coating composition further comprises a therapeutic agent.

31. The stent of claim 20, wherein the second coating composition is free of a therapeutic agent.

32. The stent of claim 20 further comprising a first therapeutic agent disposed in the pores of the non-releasable metal oxide, and wherein the second coating composition further comprises a second therapeutic agent.

33. The stent of claim 32, wherein the first and second therapeutic agents are the same.

34. The stent of claim 30, wherein the therapeutic agent comprises an anti-thrombogenic agent, anti-angiogenesis agent, anti-proliferative agent, antibiotic, anti-restenosis agent, growth factor, immunosuppressant or radiochemical.

35. The stent of claim 30, wherein the therapeutic agent comprises an anti-proliferative agent that inhibits smooth muscle cell proliferation.

36. The stent of claim 30, wherein the therapeutic agent comprises paclitaxel.

37. The stent of claim 30, wherein the therapeutic agent comprises sirolimus, tacrolimus, pimecrolimus, zotarolimus or everolimus.

38. The stent of claim 28, wherein the therapeutic agent comprises paclitaxel.

39. An implantable coated stent comprising:
(a) a substrate having a surface; and
(b) a coating disposed on at least a portion of the surface, wherein the coating comprises:
(i) a first coating composition disposed on at least a portion of the surface, wherein the first coating composition comprises a non-releasable metal oxide comprising a titanium oxide and having a plurality of pores therein; and
(ii) paclitaxel disposed in the pores of the non-releasable metal oxide; and
(iii) a second coating composition comprising paclitaxel and a releasable metal oxide disposed on at least a portion of the first coating composition, where in the releasable metal oxide comprises an adduct of a titanium oxide and an acetate, and
wherein the coating is free of any synthetic polymer that is not part of any releasable metal oxide.
